(19) United States
(12) Patent Application Publication
(10) Pub. No.: US 2013/0217983 A1
(43) Pub. Date: Aug. 22, 2013

(54) ANALYTE SENSOR

(71) Applicants: Mihailo V. Rebec, Bristol, IN (US); Slavko N. Rebec, Bristol, IN (US); Richard G. Sass, Portland, OR (US)

(72) Inventors: Mihailo V. Rebec, Bristol, IN (US); Slavko N. Rebec, Bristol, IN (US); Richard G. Sass, Portland, OR (US)

(73) Assignee: WELLSENSE, INC., Portland, OR (US)

(21) Appl. No.: 13/731,000

(22) Filed: Dec. 29, 2012

Related U.S. Application Data
(60) Provisional application No. 61/581,574, filed on Dec. 29, 2011.
(60) Provisional application No. 61/596,675, filed on Feb. 8, 2012.

(51) Int. Cl. A61B 5/1459; A61B 5/145

(52) U.S. Cl. CPC A61B 5/1459; A61B 5/14532
USPC 600/316; 600/309; 600/310; 29/428

(57) ABSTRACT

Embodiments provide analyte sensors, such as implantable analyte sensors, and methods of producing the same. An implantable sensor may include a base with a plurality of chambers. One or more sensor reagents may be retained within the chambers to form analysis regions. A membrane may be coupled to the chambers over the sensor reagents. The implantable sensor may be implanted into the dermis of a subject. One or more of the sensor reagents may exhibit a color change in response to the presence of a target analyte or reaction product thereof. The wavelengths of light reflected from the analysis regions may be detected and analyzed to determine a target analyte concentration. One or more portions of the sensor or components thereof may be configured to facilitate calibration of the sensor, correction of an optical signal obtained from the sensor by a reader device to accommodate variations in the surrounding tissues, and/or calculation of a representative value by a reader device.

### CROSS REFERENCE TO RELATED APPLICATIONS

The present application claims priority to U.S. Provisional Patent Application No. 61/581,574 filed Dec. 29, 2011 and 61/596,675 filed Feb. 8, 2012, both entitled “Analyte Sensor,” the entire disclosures of which are hereby incorporated by reference in their entireties.

### TECHNICAL FIELD

Embodiments herein relate to the field of sensors, and, more specifically, to long-term implantable micro-optical sensors.

### BACKGROUND

The continuous long-term monitoring of medical conditions such as diabetes presents challenges for both patients and medical care providers. Traditional methods that require the patient to repeatedly obtain and test blood or other fluids can be painful and inconvenient, and this may lead to reduced compliance on the part of the patient.

Implantable sensors have been developed to mitigate these drawbacks. Many of these are expensive, bulky, and require a power source or specialized reader. More recently, an optical sensor was developed with a layered hydrogel body and several analysis zones covered by a window material. Each analysis zone includes an analyte sensing reagent coupled to a pH-sensing chromogen on microbeads. While this sensor does not require a separate power source, it lacks the mechanical strength necessary to remain intact and functional for extended periods of time in the skin. The sensor can leak the contents of the analysis zones or break apart while implanted. In addition, good reflectance signals from the analysis zones can be difficult to obtain. The sensors may also be difficult to remove several weeks after implantation.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A to 1D illustrate plan views of implantable sensors in accordance with various embodiments;

FIGS. 2A to 2D illustrate side views of an implantable sensor as shown in FIG. 1a, in accordance with various embodiments;

FIG. 3 illustrates an example of a reagent system for glucose detection in an implantable sensor;

FIGS. 4A to 4C illustrate examples of sensor body configurations for use to practice various embodiments;

FIGS. 5A to 5D illustrate examples of sensor configurations;

FIG. 6 illustrates an example of an analyte monitoring system; and

FIG. 7 is a flowchart of a method for constructing an implantable sensor, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide analyte sensors, such as implantable analyte sensors, and methods of producing the same. Implantable sensors as described herein may be more robust, more easily optically read, thinner, less expensive to produce, and/or more easily removed than prior known implantable sensors.

For the purposes of this description, an “implantable sensor” is a sensor that is in planted into the skin with the main body of the sensor, or a portion thereof, residing in the dermis of the skin. In some embodiments, the entirety of the implanted sensor may reside in the dermis. In other embodiments, a portion of the implanted sensor may protrude into the epidermis, extending through the outer surface or to just below the surface of the skin. The sensor or a portion thereof may be implanted to a depth of 20 μm to 200 μm below the surface of the skin. The implantable sensor may reside in the skin for a period of time that can range from one hour to a couple of years depending upon one or more factors, such as the type(s) of analysis needed and the stability of the analysis components. The implantable sensor may be inserted and/or removed with an insertion/removal device. In some embodiments, an implantable sensor may be retained in the body of a subject for at least a minute. In other embodiments, an implantable sensor may be configured to reside in the body of a subject (e.g., in the skin) for at least one month. In still other embodiments, an implantable sensor may be configured to reside in the body of a subject for a duration of time such as a week, a month, 2-4 months, 3-6 months, or more than 6 months.

For the purposes of this description, a “response” is a change exhibited by an implantable sensor or portion thereof (e.g., in an analysis region) upon exposure to a target analyte/parameter. A “response” can be, but is not limited to, a shift in wavelengths absorbed, a shift in wavelengths deflected, an emission of light, a change in the intensity of light deflected/reflected/emitted (e.g., spectral intensity, radiant intensity spectral power, radiance, or spectral radiance), or any other measurable change in deflected/reflected/emitted light. A “response” can also be a value that is representative of such a change. In some embodiments, some or all of the wavelengths may be in the visible range. In other embodiments, some or all of the wavelengths may be in the infrared range. A “response” can be a color or color change.

For the purpose of this description, the term “subject” includes humans as well as non-human animals.

For the purpose of this description, the term “color” includes colors within the visible spectrum and colors not visible to the human eye (e.g., infrared).

In one embodiment, an implantable sensor may have a base, a body defining one or more chambers, and one or more permeability/blocking members. The base may be constructed from one or more materials such as a polymer or a metal. The body may be coupled to a surface of the base. The chambers may be one or more gaps, wells, or voids extending partially or fully through the thickness of the body. An analyte reagent system with one or more sensor reagents may be retained within a chamber. One or more permeability/blocking members may be coupled to the chambers and/or to the body, with at least some of the sensor reagents retained between the permeability/blocking member(s) and the body. In some embodiments, one or more of the sensor reagents are retained between the permeability/blocking member(s) and the base/body. The analyte reagent system may be configured to respond to the presence of an analyte by changing color and/or emitting light (luminescence). The response may be exhibited in proportion to the concentration of the analyte. In some embodiments, a response may include colors/wavelengths within the visible spectrum. In other embodiments, a response may include colors/wavelengths beyond the visible spectrum (e.g., one or more colors/wavelengths in the infrared range). For example, an analysis region may exhibit a maximum response in the infrared range. This may allow improved detection of the response, due to the lesser absorption of wavelengths within this range by the tissues surrounding the analyte sensor.

The sensor may be implanted within or below the skin of a subject. The analyte reagent system within a chamber may respond to the presence of the target analyte by producing a color change (e.g., a change in the wavelengths absorbed/reflected/emitted by the sensor). The reflected wavelengths from each analysis region may be read by a reader device such as an optical sensor (e.g., a camera). The optical data acquired by the optical sensor may be converted to an analyte concentration, such as a blood glucose value.

The sensor may have multiple analysis regions. An analysis region may include a chamber and the analyte reagent system within the chamber. Optionally, the analysis region may also include the underlying base and/or one or more permeability/blocking member(s). Thus, a first chamber may be part of a first analysis region, a second chamber may be part of a corresponding second analysis region, and a third chamber may be part of a corresponding third analysis region. An analysis region may exhibit a response, such as a change in color or electrical current generated, upon exposure to the target analyte that is present at a concentration within the range of detection of the analysis region. One or more of the analysis regions may be a reference or control region configured for use in calibration of the reader device.

The reader device may detect the responses of multiple analysis regions. In some examples, the reader device may also convert the acquired data into a representative value (e.g., a target analyte concentration, a temperature, a pressure, etc.), compare the detected responses or values to one another, compare the detected responses or values to previous responses or values, display a representative value, alert a sensor user of the representative value, and/or alert a sensor user or user of the reader device of a possible sensor malfunction.

One or more of the analysis regions may be a reference or control region(s). Reference/control regions may be configured for use in calibration of the reader device (e.g., an optical sensor) and/or correction of a measured or calculated value. For example, an analysis region may provide a reference or control that can be used by the reader device to correct for differences in circulation and/or diffusion changes. Some reference/control regions configured for use in calibration or correction of values may be parts of the sensor base, body, or other component, rather than an analysis region. Such reference/control regions may be used, for example, to determine optical corrections for differences in ambient light or light intensity, skin pigmentation/color, skin scattering, or image exposure/collection times, and/or differences in the depth of the sensor in the skin (e.g., for a sensor that is placed at a greater or lesser depth in the skin than recommended).

A control region may provide a reference, such as a color, current, or shape, for calibration of the reader device. In some examples, a control region may be a duplicate of another analysis region (i.e., may detect the same target analyte within the same range of detection and have the same range of response). The reader device may compare the responses of the two regions, and determine whether the two responses are the same within a margin of error. If a difference between the two responses is determined to exceed the margin of error, the reader device may determine that the sensor is malfunctioning. Alternatively, the reader device may average the responses from the two regions and determine a representative value for the target analyte (or non-target control analyte) based on the determined average. Optionally, the reader device may determine that a response or value from one of the two regions exceeds a predetermined threshold/value, differs from an average or other selected value by more than a predetermined limit, or is outside a particular range, such as an expected range. In response, the reader device may disregard that response or value. For example, the reader device may exclude that response/value when determining a representative value for the target analyte (or non-target control analyte).

Alternatively, a control region may detect another analyte, such as an analyte that is typically present at relatively constant levels within the dermis. Examples of such analytes include, but are not limited to, sodium, potassium, pH, creatinine, uric acid, chloride, and cholesterol. The reader device may read the control/reference region and compare the acquired data to a set of reference values (e.g., a set of previous readings or a standard set of values). If a difference between the reading and the reference values is determined to exceed a margin of error, the reader device may respond by adjusting one or more representative values (e.g., glucose values) as a function of the difference. As another example, the reader device may determine that the reading may be inaccurate, or that the sensor is malfunctioning, and/or alert the sensor user.

As still another alternative, a control region may be configured to exhibit a response to a non-target analyte, and the response may be used by the reader device to correct or determine representative values for a target analyte based on a local condition such as local blood/fluid flow. For example, a control region may be configured to detect a non-target analyte that is administered to a subject. Optionally, the non-target analyte may be administered with a drug, a treatment, or a dose of the target analyte. The time at which the non-target analyte is administered may be entered into the reader device. The reader device may read the control region continuously or at timed intervals for some period of time. The control region may exhibit a response to the non-target analyte. The reader device may correct or determine a representative value for a non-target analyte as a function of the length of time between the administration of the drug/treatment and the detection of the analyte by the control region. Optionally, the reader device may determine that the length of time exceeds a predetermined limit and alert the sensor user or reader device user of a condition such as poor circulation or possible sensor malfunction. As another option, the response time may be used to determine and/or correct for a sensor lag time, such as a difference between the length of time required for the sensor to detect an analyte (e.g., a drug, treatment, or other analyte) in the dermis and the length of time required for the analyte to be detected in an analysis of whole blood, plasma, or other fluid(s).

Implantable sensors may have one or more indicators for various purposes, such as for confirmation of sensor integrity or calibration of the reader device. These controls may be features on or within the sensor. For example, a sensor may be provided with a component or portion that has a fixed color. The reader device may adjust one or more relative values based on the difference between the color of the indicator prior to insertion and the color of the indicator after insertion in order to compensate for differences in skin tone or depth of implantation.

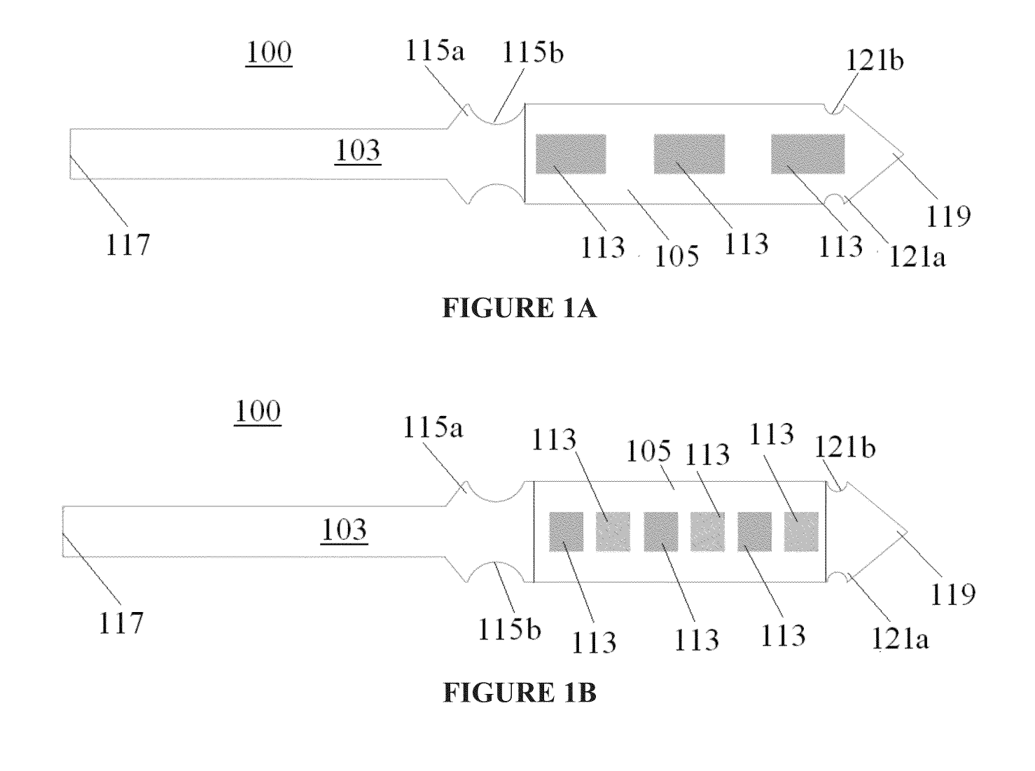

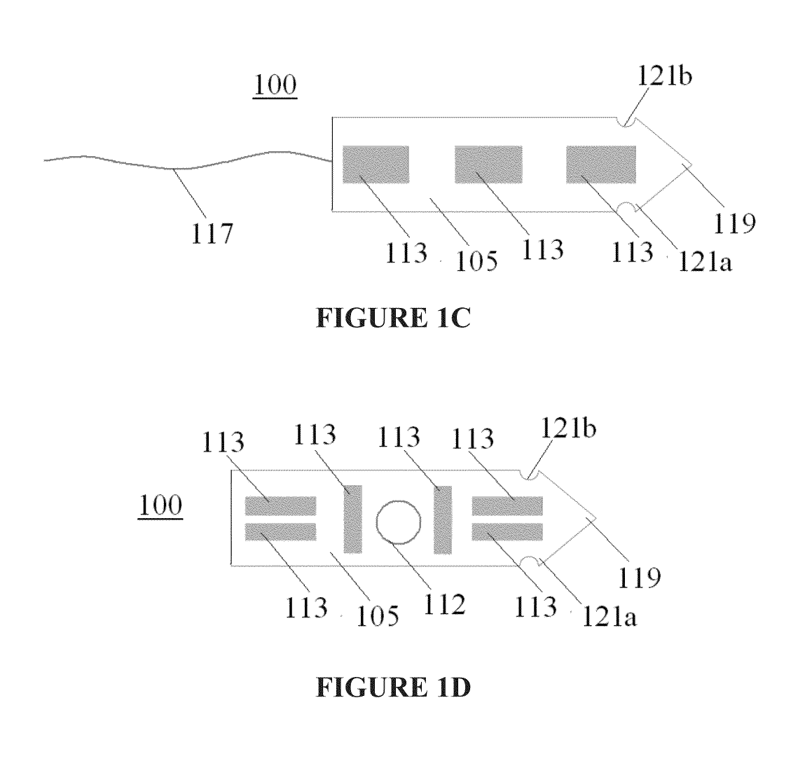

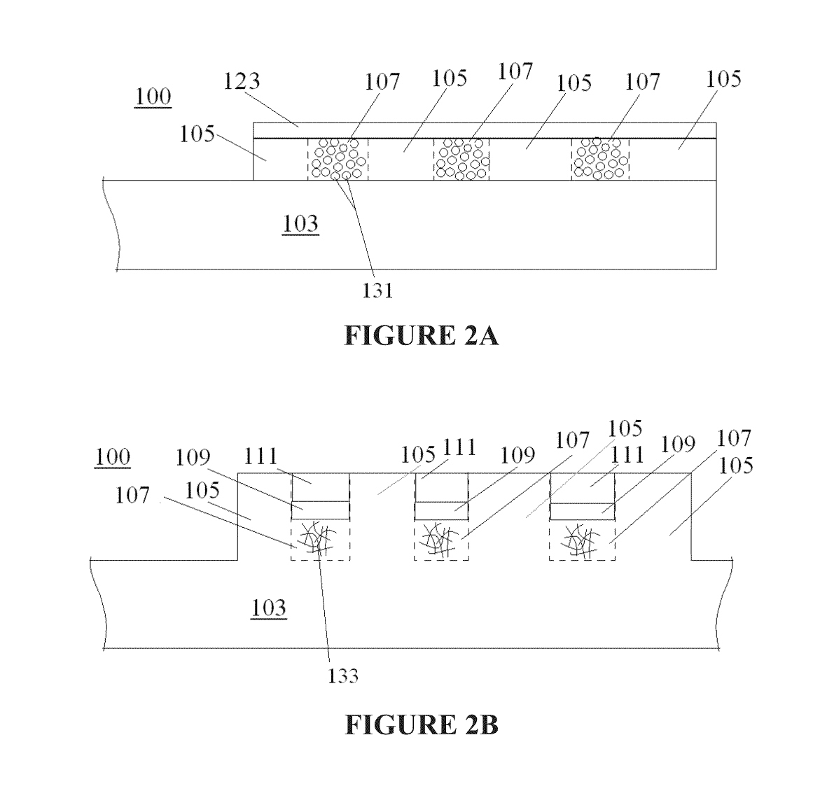

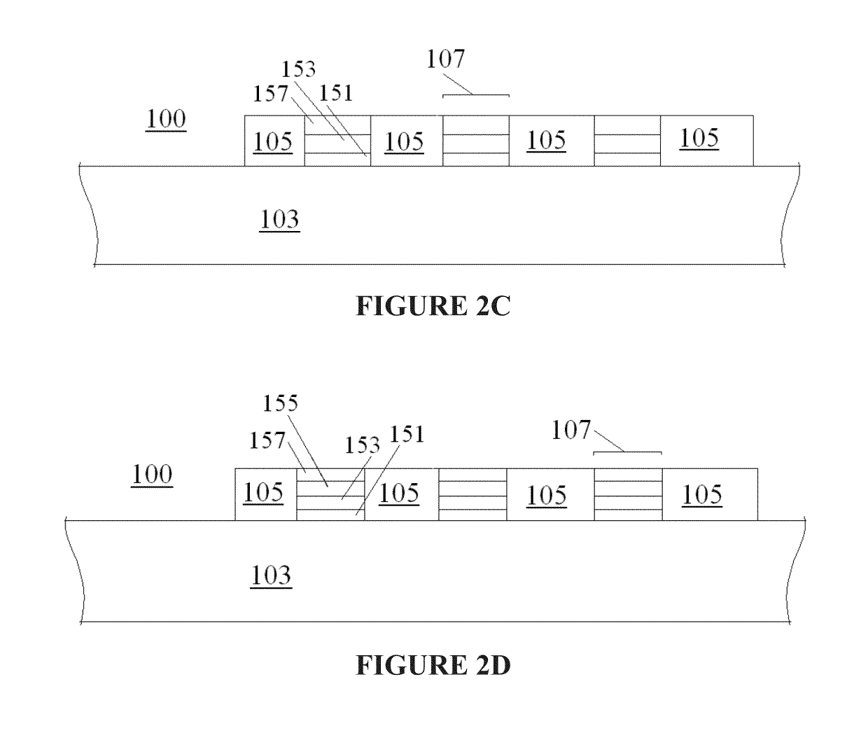

FIGS. 1A, B, C, and D illustrate plan views of implantable sensors in accordance with various embodiments. FIGS. 2A and 2B illustrate side views of an implantable sensor as shown in FIG. 1A, in accordance with various embodiments.

As illustrated, an implantable sensor 100 may have a base 103 coupled to a body 105. Analysis regions 113 may be arranged along base 103 and surrounded by body 105. Some of the analysis regions may be provided with an analyte reagent system, including one or more sensor reagents, for analyzing the target analyte(s). One or more of the other analysis regions may be configured to serve as a control for calibration and/or to confirm correct positioning, functionality, and/or accessibility of implantable sensor 100 to the target analyte(s) or control analyte(s).

Base 103 and body 105 may form first and second layers, respectively, of implantable sensor 100 (see FIG. 2A). Alternatively, body 105 and base 103 may be formed as integral portions of a single unit (see FIG. 2B). For example, body 105 and base 103 may be a single piece formed by molding, thermoforming, vacuum forming, compaction and sintering, cutting, or extrusion of a base material. Base 103 may have an elongate shape with a first end 117 and an opposite second end 119. Second end 119 may terminate in a point or other shape to aid penetration into the skin during implantation or subsequent removal of the sensor from the skin. Base 103 may include one or more surface or edge features configured to enhance the retention of implantable sensor 100 within the dermis after implantation. In the examples of FIGS. 1A and 1B, implantable sensor 100 includes projections 115a and 121a near a first end and a second opposite end, respectively, of body 105. Invaginations 115b and 121b are positioned between the projections and body 105. These features may provide resistance to backward-directed pulling forces to prevent the dislocation of the implantable sensor after implantation.

In some embodiments, second end 119 may be inserted into the dermis of a subject and first end 117 may be retained externally, above the epidermis, for removal. For example, the terminal edge (e.g., 0.5 mm) of first end 117 may protrude from the surface of the skin. In other embodiments, first end 117 may be positioned within the epidermis a short distance below the outer surface of the skin, and may become exposed for removal 1, 2, 3, 4, 5, or 6 months after implantation. In still other embodiments, first end 117 may be positioned below the epidermis after implantation. First end 117 may alternatively be positioned within the epidermis and may become exposed by natural exfoliation of the epidermis over a period of weeks or months. As another alternative, first end 117 may be inserted into the dermis of a subject and second end 119 may be retained externally (above the epidermis), within the epidermis, or below the epidermis as described above.

As shown in FIG. 1C, first end 117 may be a relatively thin and flexible member, such as a narrow tape or string, which can be grasped and pulled to remove the sensor from the skin. Other sensors may lack an elongated end. Optionally, sensors may have a surface feature configured to mate with a portion of a removal device for removal of the sensor. For example, as shown in FIG. 1D, a sensor may be provided with a hole 112 through a portion of the base and/or body. A portion of an insertion/removal device may be inserted through the hole and pulled to remove the sensor from the skin. The sensor may be configured to at least partially fold or collapse for removal. Some sensors may have a pointed or narrow end to aid in removal of the sensor from the dermis.

Base 103 can include one or more materials such as a metal and/or metal alloy (e.g., stainless steel), a hydrogel, a plastic or polymer, a biopolymer (e.g., a polyanhydride), ceramic, and/or silicon. Examples of plastics or polymers may include, but are not limited to, polyacrylic acid (PAA), cross-linked polyethylene (PEX, XLPE), polyethylene (PE), polyethylene terephthalate (PET, PETE), polyphenyl ether (PPE), polyvinyl chloride (PVC), polyvinylidene chloride (PVDC), polylactic acid (PLA), polypropylene (PP), polybutylene (PB), polybutylene terephthalate (PBT), polyamide (PA), polyimide (PI), polycarbonate (PC), polytetrafluoroethylene (PTFE), polystyrene (PS), polyurethane (PU), polyester (PEs), acrylonitrile butadiene styrene (ABS), poly(methyl methacrylate) (PMMA), polyoxymethylene (POM), polysulfone (PES), styrene-acrylonitrile (SAN), ethylene vinyl acetate (EVA), and styrene maleic anhydride (SMA).

Base 103 may have a thickness in the range of 30 μm to 500 μm. For example, base 103 may have a thickness in the range of 30-35 μm, 35-40 μm, 40-50 μm, 50-60 μm, 60-70 μm, 70-80 μm, 80-100 μm, 100-150 μm, 150-200 μm, 200-250 μm, 250-300 μm, 300-350 μm, 350-400 μm, 400-450 μm, or 450-500 μm.

In some sensors, ambient light may be reflected by reagents within chambers 107, and the resulting diffuse reflection signal may be measured by a reader device. Optionally, base 103 may include a reflective material that is integral (i.e., integrated within the material used to form base 103) or provided in the form of a coating along one or more surfaces of base 103, such as a coating along the bottom surface. The inclusion of reflective materials in or on base 103 may reduce background effects from tissue below the sensor and/or enhance the reflection or transflection of light from by the sensor. At least some ambient light may pass through the reagents within chambers 107 to be reflected by the reflective material of base 103. The resulting transflectance signal may be measured by a reader device. In such examples, the sensor may provide diffuse reflection signals and/or transflectance signals, and the reader may measure the signals of one or both types. In one example, base 103 includes a strip of polyimide material impregnated with titanium dioxide (TiO₂). Optionally, base 103 may be thicker at a first end than at a second, opposite end, to provide an optical gradient.

Body 105 may be constructed from a variety of materials depending on the strength and permeability desired. In some examples, body 105 may be a plastic or a polymer (e.g., polyimide). Body 105 may range in thickness from 5 μm to 500 μm thick. For example, body 105 may have a thickness in the range of 5-10 μm, 10-15 μm, 15-20 μm, 20-25 μm, 25-30 μm, 30-35 μm, 35-40 μm, 40-45 μm, 45-50 μm, 50-60 μm, 60-70 μm, 70-80 μm, 80-100 μm, 100-150 μm, 150-200 μm, 200-250 μm, 250-300 μm, 300-350 μm, 350-400 μm, 400-450 μm, or 450-500 μm. In one example, base 103 is a strip of polyimide material impregnated with TiO₂, and body 105 is polyurethane.

Body 105 can be applied onto base 103 as a liquid solution or vapor by printing, roll-coating, dip-coating, spin coating, spraying, chemical/physical vapor deposition, sol-gel, or other known methods. In some examples, the solution or vapor may be applied indiscriminately to an area of base 103. A pattern mask or other physical/chemical blocking agent may be used to prevent deposition of the solution or vapor over the areas where chambers 107 are desired. In other examples, the solution may be applied selectively to some areas of base 103, leaving other areas (e.g., chambers 107 and/or first end 117) untreated. Alternatively, body 105 may be a pre-formed solid, semi-solid, or gel, and may be coupled to base 103 with an adhesive. In some embodiments, body 105 and base 103 are formed as a single unit. Base 103 and/or body 105 can have varying thicknesses. Some embodiments may lack a body 105.

As best viewed in FIGS. 2A-D, one or more chambers 107 may extend partially or entirely through the thickness of body 105. Chambers 107 may be cut from body 105 before or after body 105 is applied or coupled to base 103. Alternatively, body 105 and base 103 may be a single unit, and chambers 107 may be made during formation of the unit (e.g., as part of a molding process) or after formation of the unit (e.g., by cutting or otherwise removing material from the unit).

The number, shape, depth, and spatial arrangement of chambers 107 may vary among embodiments. Similarly, the shape and depth of chambers 107 may vary within an individual sensor, with some chambers having a greater depth or different shape than others. An implantable sensor may have 2, 3, 4, 5, 6, 7, 8, 9, 10, or more than 10 chambers 107. In one example (FIG. 1A), the implantable sensor has three rectangular areas (i.e., chambers 107) that may be, for example, 800 um×400 μm in size. In another example (FIG. 1B), the implantable sensor has six rectangular areas that may be, for example, 300×400 μm in size. In other embodiments, one or more of chambers 107 may be round, oblong, polygonal, and/or have one or more tapered sides. Some embodiments may lack chambers 107.

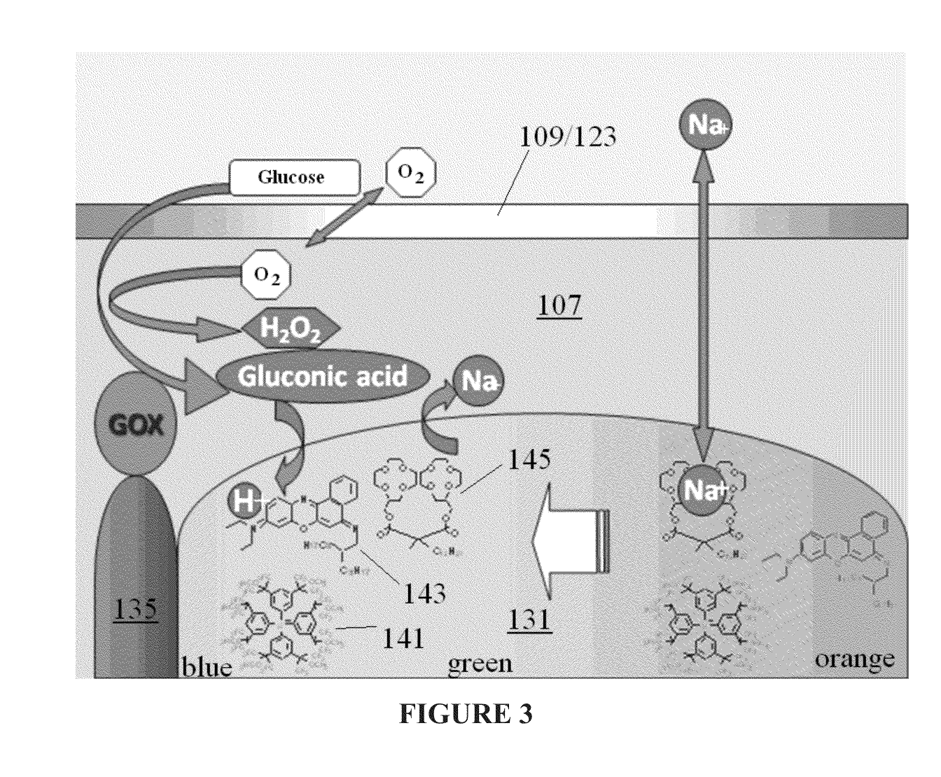

At least some of chambers 107 may contain an analyte reagent system with one or more sensor reagents, discussed further below with reference to FIG. 3. Sensor reagents may be bound to microscopic beads, fibers, membranes, gels, or other matrices in various combinations. Some sensor reagents may be retained between membranes, bound to membrane materials coated onto a membrane, or coupled/immobilized to a hydrophilic matrix. The analyte reagent system may be provided in a single layer or in multiple layers. For example, an analyte reagent system may include two, three, four, five, six, seven, eight, nine, ten, or more than ten layers.

At least one of the layers may be a permeability/blocking member, such as a membrane or gel that is selectively permeable to one or more sensor reagents, analytes, or reaction products. Examples of permeability/blocking members are described in U.S. Pat. No. 7,964,390, which is hereby incorporated by reference in its entirety. Permeability/blocking members may include one or more membranes, such as cellulose acetate membranes, cellulose acetate phosphate membranes, cellulose acetate pthalate membranes, and/or polyurethane membranes. Other permeability/blocking members may include, for example, a hydrogel, polyurethane, polyvinylpyrrolidone, acrylic polyesters, vinyl resins, fluorocarbons, silicones, rubbers, chitosan, hydroxyethylmethacrylate (HEMA), and/or polyhydroxyethylmethacrylate.

One or more of the layers may comprise a liquid or gel. In some embodiments, the liquid (or a liquid component of the gel) may be provided by the surrounding tissue after implantation of the sensor. For example, a layer may include one or more gel components in a dehydrated form, such as a powder, that is configured to form a gel upon exposure to tissue fluids.

As discussed above, other embodiments may lack a body 105. In one example, chambers may be formed in a base by pressure (e.g., forming indentations in the base material). Analysis reagents may be added into the indentations, and a layer of permeable membrane (e.g., a membrane) may be attached to the base (e.g., by an adhesive or other means) to cover the analysis reagents.

Still other embodiments may lack a body 105 and chambers 107. For example, analysis reagents may be coupled to a base, and a coating of biocompatible material may be applied to the entire sensor or to some portion thereof (e.g., over the analysis reagents). Alternatively, the base may be a flat reflective sheet of material, and analysis reagents may be deposited onto the base in predetermined shapes (e.g., dots, squares, etc.). One or more permeability components (e.g., membranes, gel, etc.) may be added over the analysis reagents. Optionally, the entire sensor may be coated with a biocompatible hydrogel. In this design there would be no thoughts, wells, etc. There would dots applied to a base.

FIG. 2C illustrates an embodiment of a sensor with a three-layer analyte reagent system. In this embodiment, the analyte reagent system includes a first layer 151, a second layer 153, and a third layer 157.

First layer 151 may include a matrix and an indicator. The matrix may include one or more of a liquid, a gel, beads, fibers, a membrane or membrane component(s), and/or another porous material. Some of the sensor reagents may be dispersed in the matrix or bound to a component thereof. The indicator may be a group of sensor reagents configured to collectively provide a response, such as a color change, upon exposure to a target analyte.

An indicator may be a pH sensitive dye that produces a color change in response to a change in pH resulting from a target analyte or reaction product/intermediate. The indicator may return to its previous color when the pH returns to its previous level. An indicator may include a group of chemical species that function as a system. For example, an indicator may include one or more of an ionophore, a lipophilic anion, and a chromoionophore (i.e., a lipophilic hydrogen ion sensitive dye). The ionophore may extract the ion to be detected (e.g., hydrogen), causing the chromoionophore to change color. Electrical neutrality may be maintained by the negatively charged anion. For example, as illustrated in FIG. 3, an indicator may include a chromogen, an ionophore, and a lipophilic anion. In other embodiments, an indicator may be a luminescent reagent that emits light in response to a target analyte or reaction product/intermediate. Luminescent reagents may include, but are not limited to, photoluminescent (e.g., phosphorescent or fluorescent), chemiluminescent, electroluminescent, electrochemiluminescent, or bioluminescent reagents. Alternatively, an indicator may be an enzyme or reaction product thereof. Some embodiments may include two or more indicators in the same or different analysis regions.

In some examples, the matrix may be a membrane and the first group of sensor reagents may be immobilized on the membrane. In other examples, at least some of the sensor reagents of the indicator may be bound to a matrix component, such as beads 131 (FIG. 2A) or elements 133 (e.g., fibers, a membrane, a membrane component, or other porous material; FIG. 2B). Different sensor reagents may be bound to separate membranes, beads, or other matrix components, or to different portions of a single membrane, bead, or matrix component.

Second layer 153 may be coupled to first layer 151. Second layer 153 may include a detection reagent. A detection reagent is a reagent that reacts with, or catalyzes a reaction of, the target analyte to produce a reaction product or intermediate. A detection reagent may be an enzyme or an enzyme system. For example, a detection reagent for glucose detection may be glucose oxidase (“GOX”), and a detection reagent for lactose detection may be lactase. In some embodiments, a detection reagent may be or include an antibody that binds to an analyte or reaction product, and/or an enzyme attached to such an antibody. The binding of the antibody to the analyte or reaction product may cause a change in the activity of the enzyme, which may influence or cause a change in pH. Thus, an analyte reagent system can include any antibody, enzyme, antibody-enzyme complex, or indicator known in the art for use in the detection of analytes in vitro or in vivo.

Second layer 153 may include a liquid, a gel, beads, fibers, a membrane or membrane component(s), and/or another porous material. In some examples, second layer 153 may include a membrane that is selectively permeable to a target analyte. The membrane may be impermeable to one or more sensor reagents (e.g., detection/indicator reagents). A detection reagent may be immobilized on a membrane, beads, or other element of second layer 153.

Third layer 157 may be a permeability/blocking member, such as a membrane, that is configured to selectively limit the passage of a target analyte or interfering compounds into second layer 153. A permeability/blocking member may include one or more membranes and/or gels, alone or in combination. Examples of permeability/blocking members are described in U.S. Pat. No. 7,964,390, which is hereby incorporated by reference in its entirety. Permeability/blocking members may include one or more membranes, such as cellulose acetate membranes, cellulose acetate phosphate membranes, cellulose acetate pthalate membranes, and/or polyurethane membranes. Other permeability/blocking members may include, for example, a hydrogel, polyurethane, polyvinylpyrrolidone, acrylic polyesters, vinyl resins, fluorocarbons, silicones, rubbers, chitosan, hydroxyethylmethacrylate (HEMA), and/or polyhydroxyethylmethacrylate.

Optionally, as shown in FIG. 2D, a fourth layer 155 may be applied to reduce or prevent damage to another layer during manufacturing. For example, fourth layer 155 may be applied over first layer 151, and second layer 153 may be applied over protective layer 155. This may protect first layer 151 from being damaged as second layer 153 is being applied.

In other embodiments, some or all of the detection reagent(s) and indicator(s) may be provided within a single layer (see e.g., FIGS. 2A, 2B, and 3). The indicator and detection reagent may be immobilized within the layer on beads, membranes, fibers, or other elements. A permeability/blocking member 109 may be coupled to the chambers 107 and/or to the body 105, and the detection reagent and indicator may be retained between the permeability/blocking member 109 and the body 105. In some examples, the detection reagent and/or indicator may be bound to the underside of the permeability/blocking member 109. Permeability/blocking member 109 may include one, two, or more two layers of membrane and/or gel. Optionally, a second permeability/blocking member 111 may be added over first permeability/blocking member 109.

Permeability/blocking members of varying configurations may be used among chambers 107 to provide increased or decreased permeability to the target analyte(s) among neighboring chambers 107. For example, a first permeability/blocking member 109 of a first chamber 107 may be more or less permeable to a target analyte than a second permeability/blocking member 109 of a second chamber 107. One or more of the permeability/blocking members may be configured for a desired permeability to a control analyte, such as sodium or potassium. Permeability/blocking members may be applied individually to chambers 107 as separate units. Alternatively, permeability/blocking member 123 may be coupled to multiple chambers 107 as a single unit, as shown in FIG. 2A.

In some embodiments, individual permeability/blocking members 109 may be coupled to corresponding chambers 107, and a single permeability/blocking member 123 may be applied as a single layer across the upper surface of body 105 (see FIG. 2B). Permeability/blocking member 123 may have different configurations at different locations along its length, such as differences in pore size(s), thickness, or other parameters. This may provide neighboring chambers 107 with different permeabilities to a target analyte or reagent.

One or more of the permeability/blocking members and chambers may be made of a set of materials with a composition that varies in that permeability from one portion to another. For example, a permeability/blocking member and/or chamber can have a decrease in permeability from the upper surface to a lower portion, such that larger molecules can permeate the upper part with limited or no entry into the lower portion, but smaller molecules such as sodium and hydrogen ions can permeate the lower portion. This could be accomplished changing the relative amounts of the polymers, cross-linking agents, and/or photoinitiators that are used or deposited in the formation of the component. Therefore, in some sensors, a diffusion gradient may be provided by a single-layer permeability/blocking member or other single-layer component.

As discussed above, an analyte reagent system may include an indicator that provides a color change in response to a target analyte. An indicator may be, but is not limited to, a pH-sensitive dye with one or more chromoionophores, lipophilic anions, and/or ionophores. Other indicators may include luminescent reagents, enzymes, and/or reaction products.

Examples of chromoionophores include, but are not limited to: chromoionophore I (9-(diethylamino)-5-(octadecanoylimino)-5H-benzo[a]phenoxazine) designated ETH5249; chromoionophore II (9-dimethylamino-5-[4-(16-butyl-2,14-dioxo-3,15 ioxaeicosyl)phenylimino]benzo[a]phenoxazine) designated ETH2439; chromionophore III (9-(diethylamino)-5-[(2-octyldecyl)imino]benzo[a]phenoxazine), designated ETH 5350; chromoionophore IV (5-octadecanoyloxy-2-(4-nitrophenylazo)phenol), designated ETH2412; chromoionophore V (9-(diethylamino)-5-(2-naphthoylimino)-5H-benzo[a]phenoxazine); chromoionophore VI (4′,5′-dibromofluorescein octadecyl ester) designated ETH7075; chromoionophore XI (fluorescein octadecyl ester) designated ETH7061; and combinations thereof.

Examples of lipophilic anions include, but are not limited to: KTpCIPB (potassium tetrakis(4-chlorophenyl)borate), NaHFPB (sodium tetrakis[3,5-bis(1,1,3,3,3-hexafluoro-2-methoxy-2-propyl)phenyl]borate), sodium tetrakis[3,5-bis(trifluoromethyl)phenyl]borate, sodium tetrakis(4-fluorophenyl)borate, combinations thereof, and the like.

Examples of ionophores include, but are not limited to: Sodium ionophores, such as bis[(12-crown-4)methyl]2-dodecyl-2-methylmalonate, designated ETH227; N,N′,N″-triheptyl-N,N′,N″-trimethyl4,4′,4″-propylidynetris(3-oxabutyramide), designated ETH157; N,N′-dibenzyl-N,N′-diphenyl-1,2-phenylenedioxydiacetamide, designated ETH2120; N,N,N′,N′-tetracyclohexyl-1,2-phenylenedioxydiacetamide, designated ETH4120; 4-octadecanoyloxymethyl-N,N,N′,N′-tetracyclohexyl-1,2-phenylenedioxydiacetamide), designated DD-16-C-5; 2,3:11,12-didecalino-16-crown-5), bis(benzo-15-crown-5), and combinations thereof; Potassium ionophores, such as: bis[(benzo-15-crown-5)-4′-methyl]pimelate, designated BME 44; 2-dodecyl-2-methyl-1,3-propanedil bis[N-{5′-nitro(benzo-15-crown-5)-4′-yl]carbamate], designated ETH1001; and combinations thereof; Calcium ionophores, such as: (−)-(R,R)—N,N′-bis-[11-(ethoxycarbonyl)undecyl]-N,N′-4,5-tetramethyl-3,6-dioxaoctane-diamide), designated ETH129; N,N,N′,N′-tetracyclohexyl-3-oxapentanediamide, designated ETH5234; N,N-dicyclohexyl-N′,N′-dioctadecyl-3-oxapentanediamide), designated K23E1; 10,19-bis[(octadecylcarbamoyl)methoxyacetyl]-1,4,7,13,16-pentaoxa-10,19-diazacycloheneicosane), and combinations thereof.

FIG. 3 illustrates an example of a reagent system with a pH-sensitive indicator for use in an implantable sensor. This reagent system provides a GOx/pH based reaction that produces a color shift (i.e., a variation in reflected wavelengths of light) that can be measured to determine a glucose concentration. In this example, the chromoionophore is chromionophore III, the ionophore is bis[(12-crown-4)methyl]2-dodecyl-2-methylmalonate, and the lipophilic anion is sodium tetrakis[3,5-bis(1,1,1,3,3,3-hexafluoro-2-methoxy-2-propyl)phenyl]borate trihydrate. In this system, the chromoionophore exhibits a pH-dependent color between the extremes of orange and blue. The pH shifts in response to varying concentrations of glucose. The reflected wavelengths (orange, yellow, green, blue) from the analysis regions can be detected and analyzed to determine the local glucose concentration.

As illustrated, glucose and oxygen enter chamber 107 through permeability/blocking membrane (109/123). Chamber 107 may include an indicator coupled to a substrate 131. In the illustrated example, the indicator includes a chromoionophore 143, an ionophore 145, and a lipophilic anion 141. A detection reagent (e.g., GOx) may be immobilized on a substrate 135. Each of substrates 131 and 135 may be an independent component such as a bead, a membrane, a fiber, or a surface of body 105 that is exposed within chamber 107. In other examples, a substrate 131 and a substrate 135 may integrated within one component.

The GOx converts glucose and oxygen to gluconic acid and hydrogen peroxide. Increasing production of gluconic acid causes a shift in pH. The chromoionophore 143 accepts a hydrogen ion, which causes a shift in the color of the chromoionophore 143 toward blue. As electrical neutrality is maintained by the lipophilic anion 141, the ionophore 145 responds to the acceptance of the hydrogen ion by releasing a sodium ion to maintain the charge balance. As the production of gluconic acid decreases, the ionophore accepts a sodium ion, and the chromoionophore releases a hydrogen ion, causing a shift in color of the chromoionophore toward orange. The shift in color causes a corresponding shift in wavelengths reflected by the analysis regions, which can be detected to monitor glucose levels at desired time intervals.

Optionally, one or more additional reagents may be provided within chamber 107. The additional reagent(s) may be provided to increase the rate of a chemical reaction, stabilize one or more components of the analyte reagent system, and/or convert a reaction product to another product. For example, catalase may be provided to convert hydrogen peroxide to water and oxygen.

In some embodiments, sensor reagents of an analyte system may be segregated within chamber 107. This may be useful where two or more sensor reagents are incompatible or require different pH levels for optimal enzyme activity or stability. For example, within chamber 107, one or more pH sensing areas with an indicator may be segregated from one or more enzyme areas with detection reagents. The sensor reagents may be deposited separately in the respective areas, such as in one or more gels or on separate substrates. The respective areas may be in direct contact. Alternatively, another substrate or material may provides a transition zone between the areas. For example, a detection reagent such as GOx may be deposited in a first (enzyme) area and an indicator may be deposited in a second (pH sensing) area. Hydrogen ions generated in the reaction area would diffuse to the pH sensing area. Optionally, the hydrogen ions could diffuse through a hydrogel disposed between the two areas. While some examples may have only two separate areas, others may have any number of micro-areas dispersed throughout chamber 107 or portions thereof. Multiple areas or micro-areas may be disposed in one or more patterns. Examples of suitable patterns include, but are not limited to, alternating dots/squares/lines and concentric circles. In a specific example, two respective areas are arranged to form two or more separate, concentric circular portions, with one of the areas (e.g., an enzyme area) disposed in an outer ring and surrounding the other area (e.g., a pH-sensing area).

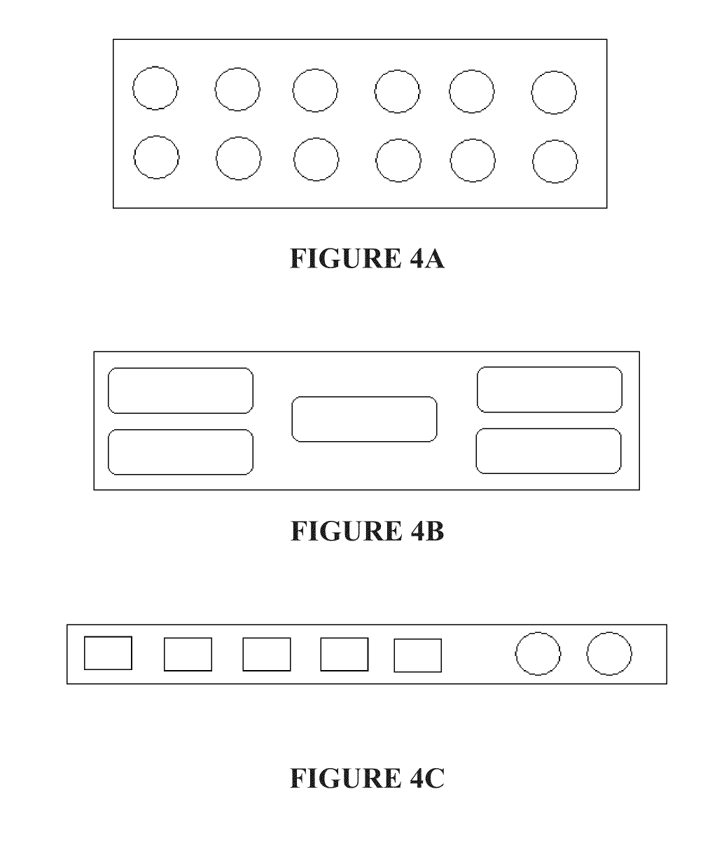

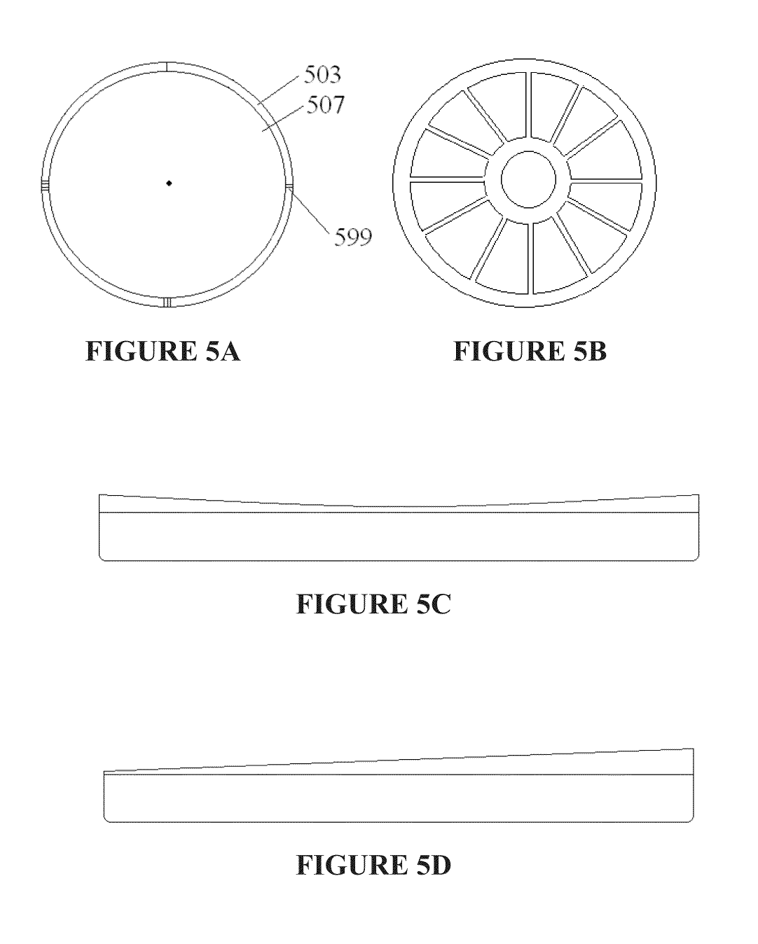

FIGS. 4A-4C and FIGS. 5A-5D illustrate examples of sensor body configurations for use to practice various embodiments. As illustrated in these FIGS. 4A-4C, a sensor may include chambers of any number, size, and arrangement. Examples of chamber arrangements are shown in FIGS. 4A-4C. As shown in FIGS. 5A-5D, some sensors may be circular or round with a number of analysis areas located in wedges, rings, or other patterns. Some of those areas may be subdivided into areas responsive to different analyte concentration ranges. For example, a round sensor may have two or more analysis regions arranged in concentric rings. The inner ring may be configured to exhibit a response to an analyte concentration that is within a first range, and the second ring may be configured to exhibit a response to an analyte concentration that is within a second range. Alternatively, one or both of the rings may be configured for use as a control region and/or for detecting a non-target analyte.

A substantially round sensor may have a single continuous chamber (FIG. 5A) or multiple chambers (FIG. 5B). The sensor may optionally include a permeability/blocking member that is configured to provide a permeability gradient, as discussed above. In some sensors, a permeability gradient may be provided by a permeability/blocking member that is thinner at the center than at the outer edge (FIG. 5C) or is thinner at one side than at another side (FIG. 5D).

Referring to FIG. 5A by way of example, a sensor may have one or more orientation marks 599. Orientation marks 599 may have any suitable shape, size, color, or location, and may be provided on any component or portion of an implantable sensor (e.g., to base/body 503, chamber 507, a permeability/blocking member, and/or any other component). Orientation members 599 may be used by a reader device to determine the location of an analysis region on the sensor, to orient a captured image with regard to a reference image or pre-determined pattern, and/or to select an area for image capture. The reader device may determine a detection range for an analysis region based on its position relative to one or more of orientation marks 599, the center or edge of the sensor, and/or another feature of the sensor. While FIG. 5A shows orientation marks 599 on a sensor assembly with a single continuous chamber, orientation marks may be provided on sensors of any shape or configuration and used in a variety of ways. For example, the user or the reader device may view the orientation marks to confirm that the sensor is inserted in the correct orientation or to a recommended depth. Orientation marks may also provide a calibration standard for the reader device to assess, and compensate for, variations in skin tone, skin translucence, or implantation depth.

A reader device may capture an image of the sensor and analyze the image to determine one or more representative values for the target analyte(s). Here, the reader device may select and/or analyze one or more analysis regions based on color, intensity of emission, distance from the center/edge of the base/body, orientation on the sensor, prior readings, programmed instructions, and/or a pre-determined pattern or reference image. For example, the reader device may select analysis regions in a captured or real-time image based at least on a pre-determined pattern of areas for analysis. Optionally, the reader device may increase or decrease the size of a selected area based on image resolution (e.g., select a larger area where image resolution falls below a minimum threshold). The reader device may access a look-up table or database that provides detection ranges for some or all of the pre-determined analysis regions and calculate a representative value for an analyte based on the image data and corresponding detection ranges. In some examples, the reader device may select an analysis region that differs from a pre-determined analysis region in size/area, contour, and/or location. The reader device may extrapolate a detection range for this analysis region based at least on the difference(s), the pre-determined pattern, and the corresponding detection ranges provided in the look-up table or database.

Optionally, the reader device may compare two, three, or more than three selected areas or analysis regions to determine whether a portion of the sensor is exhibiting a response that is inconsistent with the response of another portion of the sensor. The inconsistency may be, for example, a difference in response time, a difference in color, or a difference in intensity. The reader device may use the comparison to determine that the sensor is leaking or otherwise malfunctioning, determine a time frame for replacement of the sensor, or engage in error correction or data smoothing to determine a representative value.

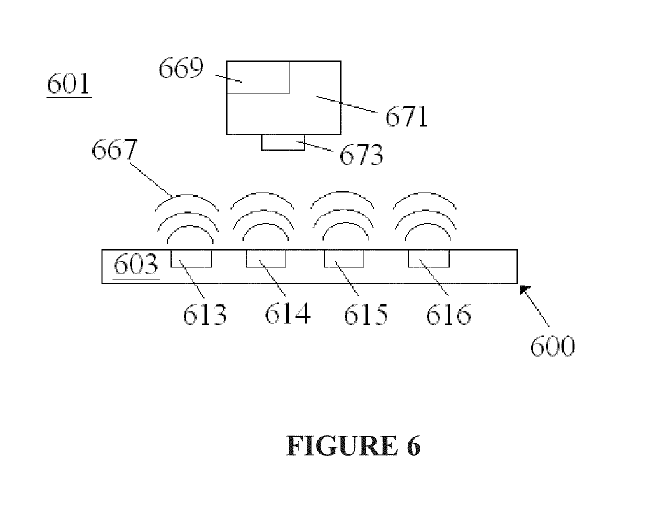

FIG. 6 shows a block diagram of an analyte detection system in accordance with various embodiments. An analyte detection system 601 may include an implantable sensor 600 and a reader device 671. Implantable sensor 600 may be configured with one or more modifications of the analyte reagent system(s), permeability/blocking member(s), base, and/or body to provide analysis regions with contiguous detection ranges and coextensive ranges of response. Sensor 600 may be configured to be implanted at least partially within the dermis of a subject.

Reader device 671 may include an optical sensor 673 and a non-transitory computer-readable storage medium 669. Optical sensor 673 may be configured to detect electromagnetic radiation 667 reflected, deflected, or emitted from sensor 600. Reader device 671 may analyze the detected responses of analysis regions 613, 614, 615, and 616 to one or more target/control analytes as discussed above. Non-transitory computer-readable storage medium 669 may be programmed with an algorithm to determine a representative value of a target analyte based at least in part on the wavelengths or intensity of electromagnetic radiation detected from two or more of the analysis regions. Optionally, reader device 671 may be a mobile device such as a camera, a PDA, a laptop, a tablet, or a wireless/cellular phone. Alternatively, the sensor may be read visually by a user without a separate reader device.

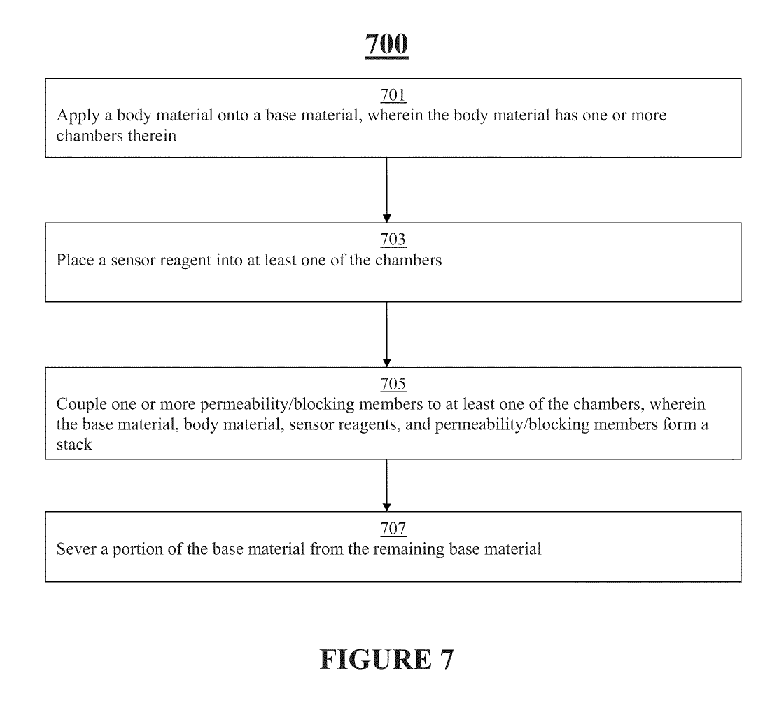

Implantable sensors can be constructed individually or in bulk according to known methods. FIG. 7 illustrates a method for constructing an implantable sensor, in accordance with various embodiments.

Method 700 may begin with block 701. At block 701, a body material may be applied onto a base material. The body material may include one or more chambers extending partially or fully through the thickness of the body material. For example, the body material may be printed discontinuously onto the base material such that one or more unprinted areas form chambers in the body material. Printing may include any one or more known printing techniques, such as screen printing or inkjet printing. As described above, the body material can be a plastic or polymer (e.g., polyurethane), and the base material may be a polyimide material impregnated with titanium dioxide. The screen printing may be discontinuous across the surface of the polyimide material to leave unprinted areas for chambers (i.e., chambers 107). The body material may be screen printed onto a portion of the sheet of material that corresponds to half or less than half the length of the base material (see e.g., FIGS. 1A and 1B and FIGS. 2A and 2B). Alternatively, unprinted areas may be formed by removing portions of the body material to form chambers.

From block 701, method 700 may proceed to block 703. At block 503, one or more sensor reagents may be placed into one or more of the chambers. Sensor reagents may be deposited in the form of a gel, liquid, solid, or semi-solid containing one or more membranes, beads, fibers, and/or other matrices to which at least some of the sensor reagents are bound. Sensor reagents may be deposited into the chambers by known methods such as screen printing, inkjet deposition, or micro deposition with nano or micro volume liquid deposition systems.

From block 703, method 700 may proceed to block 705. At block 705, one or more permeability/blocking members may be coupled to the chamber(s). One or more of the base material, body material, sensor reagents, and permeability/blocking members may form a stack.

From block 705, method 700 may optionally proceed to block 707. At block 707, a first portion of the base material may be separated from a second portion of the base material to produce individual sensors. In some embodiments, the base material and overlying layers may be assembled in one or more large sheets or strips and individual sensors may be cut from the sheets or strips. Individual sensors may be cut before addition of the sensor reagents to the base and body, and the sensor reagents and permeability/blocking member(s) may be added to individual sensors. Alternatively, individual sensors may be cut after addition of the sensor reagents and permeability/blocking members. In some examples, a portion of the base material (and optionally, the overlying portions of the body material and/or permeability/blocking member) may be separated by cutting through the base material (e.g., by laser cutting or other known methods). The sensors can be cut from the stack or sheet in various shapes in order to provide surface features for retention after insertion. The sensors can also be cut in various lengths to accommodate different placement sites and/or skin thicknesses.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An analyte sensor, comprising:
a base having an upper surface and an opposite lower surface, a first end, and a second end;
an analyte reagent system coupled to the base, wherein the analyte reagent system is configured to cause a shift in pH in response to a target analyte, and to exhibit a reversible color change in response to the shift in pH; and
one or more membranes coupled to the base, the one or more membranes being permeable to a target analyte.

2. The analyte sensor of claim 1, wherein the analyte sensor has a total thickness of 50 μm or less.

3. The analyte sensor of claim 1, wherein the base comprises a polymer and TiO₂.

4. The analyte sensor of claim 3, wherein the polymer is polyimide.

5. The analyte sensor of claim 1, wherein the base material is a highly reflective material.

6. The analyte sensor of claim 1, wherein the analyte sensor is configured to be retained within the body of a subject.

7. The analyte sensor of claim 1, wherein the analyte sensor is configured to be retained within the dermis of a subject for at least one month.

8. The analyte sensor of claim 1, wherein the base has a variable thickness, a first portion of the base being thicker than a second portion of the base, wherein the variable thickness provides an optical gradient.

9. The analyte sensor of claim 1, further comprising an elongated portion configured to be retained within or above the epidermis to aid removal of the analyte sensor from the dermis of a subject.

10. The analyte sensor of claim 9, wherein the elongated portion is an end portion of the base or a flexible member coupled to the base.

11. The analyte sensor of claim 1, wherein the base defines one or more chambers therein.

12. The analyte sensor of claim 11, wherein the analyte reagent system is disposed in at least one of the one or more chambers.

13. The analyte sensor of claim 11, wherein the base includes a body portion and a base portion, the body portion defining the one or more chambers, wherein the base comprises a reflective material.

14. The analyte sensor of claim 13, wherein the body portion consists essentially of polyurethane.

15. The analyte sensor of claim 1, further including a biocompatible coating coupled to the base.

16. The analyte sensor of claim 1, the analyte sensor having one or more orientation marks configured for use by a reader device to orient a captured image of the analyte sensor within the dermis of a subject.

17. An analyte detection system for monitoring the concentration of a target analyte in a subject, the system comprising:
a substantially flat, elongate analyte sensor configured to be retained within the body of the subject and to exhibit a reversible color change in response to a change in concentration of the target analyte; and
a reader device having an optical sensor, a processor, and a non-volatile computer-readable storage medium endowed with instructions operable, upon execution by the processor, to
capture an image of the analyte sensor within the dermis of the subject, and
calculate the concentration of the target analyte based at least on the captured image.

18. The system of claim 17, wherein the analyte sensor is configured to be retained within the dermis of the subject.

19. The system of claim 18, wherein the analyte sensor is configured to be retained within the dermis for at least one month.

20. The system of claim 17, wherein the reader device is a mobile electronic device selected from the group consisting of a laptop, a tablet PC, or a mobile phone.

21. The system of claim 20, wherein the analyte sensor further comprises an elongated base with at least a first and a second analysis region, a first analyte reagent system coupled to the first analysis region, and a second analyte reagent system coupled to the second analysis region, the first analyte reagent system configured to cause a shift in pH in response to the change in concentration of the target analyte, and to exhibit the reversible color change in response to the shift in pH.

22. The system of claim 21, wherein the first analyte reagent system includes a detection reagent and an indicator.

23. The system of claim 17, wherein the base comprises a polymer and TiO₂.

24. The system of claim 23, wherein the polymer comprises a polyimide.

25. The system of claim 22, wherein the indicator comprises two or more of a lipophilic anion, a chromoionophore, and an ionophore.

26. The system of claim 22, wherein the detection reagent comprises an enzyme.

27. The system of claim 17, wherein the analyte sensor has a total thickness of 50 μm or less.

28. The system of claim 22, wherein the detection reagent comprises glucose oxidase.

29. The system of claim 21, wherein the second analyte reagent system is configured to cause a shift in pH in response to a change in concentration of a second analyte, and to exhibit a reversible color change in response to the shift in pH.

30. The system of claim 17, the analyte sensor having one or more control regions configured for use to calibrate the reader device or to correct a representative value.

31. The system of claim 21, the analyte sensor having one or more orientation marks configured for use by the reader device to orient a captured image of the analyte sensor.

32. A method of manufacturing implantable analyte sensors, the method comprising:
forming a base;
depositing a first sensor reagent system onto the base, the first sensor reagent system configured to exhibit a reversible color change in response to a first analyte concentration;
depositing a second sensor reagent system onto the base, the second sensor reagent system configured to exhibit a reversible color change in response to a second analyte concentration;
coupling a permeability member to the body over the first and second sensor reagent systems to form a stack, wherein the permeability member is permeable to a target analyte; and
cutting the stack into two or more portions, wherein each portion includes at least one chamber of the first group and at least one chamber of the second group.

33. The method of claim 32, wherein forming the base comprises printing a body material onto a substantially flat strip or sheet of base material to form one or more chambers, and wherein the first and second sensor reagent systems are deposited into the one or more chambers.

34. The method of claim 33, wherein the base material comprises a polymer and TiO₂.

35. The method of claim 33, wherein the body material is a polymer.

36. The method of claim 33, wherein the body material is printed discontinuously onto the base material to form the chambers.

37. The method of claim 33, wherein the body material is printed continuously onto the base material, and the chambers are formed by removing portions of the body material.

38. A method of monitoring a concentration of a target analyte in a subject, the method comprising:
positioning an analyte sensor within the dermis of the subject, wherein the analyte sensor includes a first analysis region configured to exhibit a reversible color change in response to a change in concentration of the target analyte;
capturing an image of the analyte sensor with a reader device, wherein the reader device is a personal electronic device that comprises an optical sensor, a storage medium, and a processor; and
determining by the reader device, based at least on the captured image, the concentration of the target analyte.

39. The method of claim 38, wherein the reader device is a mobile electronic device selected from the group consisting of a laptop, a tablet PC, or a mobile phone.

40. The method of claim 38, wherein one or more orientation marks are provided on the analyte sensor.

41. The method of claim 40, further including locating, by the reader device, the first analysis region based on the one or more orientation marks.

42. The method of claim 40, further including determining, by the reader device, a detection range for the first analysis region based at least on the one or more orientation marks.

43. The method of claim 40, further including assessing by the reader device, based at least on the one or more orientation marks, a variation in analyte sensor implantation depth, skin tone, or skin translucence, wherein determining the concentration of the target analyte further comprises adjusting the concentration to compensate for said variation.

44. The method of claim 36, wherein the analyte sensor includes a second analysis region configured to exhibit the reversible color change in response to the concentration of the target analyte, and determining the concentration of the target analyte includes comparing responses from the first and second analysis regions.
